(19) United States
(12) Patent Application Publication
(10) Pub. No.: US 2026/0115411 A1
(43) Pub. Date: Apr. 30, 2026

(54) SLEEP ASSISTANCE SYSTEM

(71) Applicant: Panasonic Holdings Corporation, Osaka (JP)

(72) Inventors: Taizou AOKI, Kyoto (JP); Ryou TOSHIMA, Nara (JP); Shima OKADA, Osaka (JP); Hazuki MASUDA, Shiga (JP); Masanobu MANNO, Osaka (JP)

(21) Appl. No.: 19/431,058

(22) Filed: Dec. 23, 2025

Related U.S. Application Data
(63) Continuation of application No. PCT/JP2024/024136, filed on Jul. 3, 2024.

(30) Foreign Application Priority Data
Jul. 11, 2023 (JP) 2023-113490
Mar. 27, 2024 (JP) 2024-051031

(51) Int. Cl. A61M 21/02; A61M 21/00

(52) U.S. Cl. CPC A61M 21/02; A61M 2021/0022; A61M 2021/0066; A61M 2205/3334; A61M 2205/3368; A61M 2205/3379; A61M 2230/06; A61M 2230/63

(57) ABSTRACT

A sleep assistance system includes: an air blower that blows air at an extremity of a person who is sleeping; and a controller that controls the air blown by the air blower to facilitate lowering of a core temperature of the person sleeping in bedding.

### CROSS REFERENCE TO RELATED APPLICATIONS

This is a continuation application of PCT International Application No. PCT/JP2024/024136 filed on Jul. 3, 2024, designating the United States of America, which is based on and claims priority of Japanese Patent Application No. 2023-113490 filed on Jul. 11, 2023 and Japanese Patent Application No. 2024-051031 filed on Mar. 27, 2024. The entire disclosures of the above-identified applications, including the specifications, drawings and claims are incorporated herein by reference in their entirety.

### FIELD

The present disclosure relates to a sleep assistance system.

### BACKGROUND

Conventionally, a technique to control an environment in a bed for facilitating good-quality sleep is known. For example, Patent Literature (PTL) 1 discloses an environmental control system that is an environmental control system that controls an environment in a bed by air blown from an air blowing device, and includes a sleep position detection means that detects a sleep position of a user and a plurality of hygrothermal sensors. Based on a detection result of the sleep position of the user, the environment control system in PTL 1 identifies a hygrothermal sensor that is in close proximity of the user, and controls air blowing operation of the air blowing device based on a detection result of the hygrothermal sensor identified.

### CITATION LIST

### Patent Literature

PTL 1: Japanese Unexamined Patent Application Publication No. 2022-131849

### SUMMARY

### Technical Problem

In the technique disclosed in PTL 1, in order to facilitate good-quality sleep, the environment in the bed needs to be controlled based on a detection result of the hygrothermal sensor.

The present disclosure provides a sleep assistance system that can enhance the quality of sleep of a person in an efficient manner.

### Solution to Problem

The sleep assistance system according to one aspect of the present disclosure includes: an air blower that blows air at an extremity of a person who is sleeping; and a controller that controls the air blown by the air blower to facilitate lowering of a core temperature of the person.

### Advantageous Effects

The present disclosure can enhance the quality of sleep of a person in an efficient manner.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a diagram of an outline for describing a sleep assistance system according to an embodiment.

FIG. 2A is a diagram illustrating an inside of bedding illustrated in FIG. 1.

FIG. 2B is another diagram illustrating the inside of the bedding.

FIG. 3 is a block diagram illustrating a configuration of the sleep assistance system according to the embodiment.

FIG. 4 is a drawing schematically illustrating change in a core temperature when air is blown at a foot of a person who is sleeping in the bedding.

FIG. 5 is a flowchart illustrating an example of operation of the sleep assistance system according to the embodiment.

FIG. 6 is a drawing schematically illustrating a relationship between air volume of an air blower, a heat dissipation function of a person, a rate of change of an extremity temperature, and a bodily sensation when a person is sleeping.

FIG. 7 is a block diagram illustrating a configuration of a sleep assistance system according to Variation 1 of the embodiment.

FIG. 8 is a flowchart illustrating an example of operation of the sleep assistance system according to Variation 1 of the embodiment.

FIG. 9 is a block diagram illustrating a configuration of a sleep assistance system according to Variation 2 of the embodiment.

FIG. 10 is a flowchart illustrating an example of operation of the sleep assistance system according to Variation 2 of the embodiment.

FIG. 11 is a diagram for describing an example of air temperature control performed by the controller.

FIG. 12 is a flowchart illustrating another example of operation of the sleep assistance system according to Variation 2 of the embodiment.

### DESCRIPTION OF EMBODIMENT

### (Outline of Present Disclosure)

Hereinafter, an example of a sleep assistance system according to the present disclosure will be described as an outline of the present disclosure.

A sleep assistance system according to a first aspect of the present disclosure includes: an air blower that blows air to cause the air to directly hit an extremity of a person; and a controller that controls the air blown by the air blower to facilitate lowering of a core temperature of the person.

The inventors discovered that it is possible to significantly decrease the core temperature of a person by controlling air that is blown at a foot of the person who is sleeping. Accordingly, in the sleep assistance system according to this aspect, it is possible to enhance the quality of sleep of the person by facilitating the lowering of the core temperature of the person who is sleeping by simply blowing air at a foot of the person, and there is no need to control environmental factors, such as temperature, humidity, and the like in a bed. Thus, in the sleep assistance system according to this aspect, the quality of sleep of the person can be enhanced in an efficient manner.

Furthermore, for example, a sleep assistance system according to a second aspect of the present disclosure is the sleep assistance system according to the first aspect, in which the controller controls, based on the core temperature of the person, the air blown by the air blower.

Accordingly, the air blown by the air blower can be adjusted in accordance with the core temperature of the person, and the core temperature of the person can be more effectively decreased, thereby further enhancing the quality of sleep of the person.

Furthermore, for example, a sleep assistance system according to a third aspect of the present disclosure is the sleep assistance system according to the second aspect, in which the controller controls the air blown by the air blower to cause a rate of decrease of the core temperature of the person to be greater than or equal to a predetermined threshold value.

Accordingly, the core temperature of the person can be decreased at a desired rate of speed by setting a predetermined threshold value.

Furthermore, for example, a sleep assistance system according to a fourth aspect of the present disclosure is the sleep assistance system according to the second aspect or the third aspect, in which the controller obtains the core temperature of the person measured by a core temperature measurer, and controls the air blown by the air blower based on the core temperature obtained.

Accordingly, the core temperature of the person that is directly obtained can be used in controlling the air blown by the air blower, and the accuracy of control performed on the air blown by the air blower can be enhanced.

Furthermore, for example, a sleep assistance system according to a fifth aspect of the present disclosure is the sleep assistance system according to the second aspect or the third aspect, in which the controller obtains a heart rate of the person, estimates the core temperature of the person based on the heart rate obtained, and controls the air blown by the air blower based on the core temperature estimated.

Accordingly, since a measurable heart rate is easily measured, and the core temperature is estimated from the heart rate measured, information on the core temperature can be more easily obtained.

Furthermore, for example, a sleep assistance system according to a sixth aspect of the present disclosure is the sleep assistance system according to any one of the first aspect to the fifth aspect, in which the controller obtains an extremity temperature of the person, and controls an air volume of the air blower to maximize the air volume of the air blower to an extent that the extremity temperature of the person does not decrease.

Since a person is more prone to feeling cold when their extremity temperature decreases at the same time that their core temperature is low, by controlling the air volume in the manner described above, it is possible to more readily facilitate the lowering of the core temperature in a manner such that the person does not feel cold.

Furthermore, for example, a sleep assistance system according to a seventh aspect of the present disclosure is the sleep assistance system according to any one of the first aspect to the sixth aspect, in which the controller controls an air volume of the air blower to cause the air volume during a predetermined period of time starting from when the person goes to bed to be larger than the air volume of the air blower when the person wakes up.

Accordingly, since this further facilitates the lowering of the core temperature of the person, during a predetermined period of time starting from when the person goes to bed, which is a period that greatly influences the quality of sleep of the person, the quality of sleep of the person can be effectively enhanced.

Furthermore, for example, a sleep assistance system according to an eighth aspect of the present disclosure is the sleep assistance system according to any one of the first aspect to the seventh aspect, in which the controller obtains bodily sensation information that indicates a bodily sensation of the person, and controls the air blown by the air blower based on the bodily sensation information.

Accordingly, since the air blown by the air blower is controlled in accordance with bodily sensations, which are sensations of feeling hot or cold or the like, of the person who is sleeping, it becomes possible to allow the person who is sleeping to comfortably sleep, thereby further enhancing the quality of sleep of the person.

Furthermore, for example, a sleep assistance system according to a ninth aspect of the present disclosure is the sleep assistance system according to the eighth aspect, in which the bodily sensation information indicates whether the person is feeling cold.

Accordingly, since the air that is blown by the air blower is controlled according to whether the person who is sleeping is feeling cold, it becomes possible to prevent the person from feeling cold, thereby further enhancing the quality of sleep of the person.

Furthermore, for example, a sleep assistance system according to a tenth aspect of the present disclosure is the sleep assistance system according to the ninth aspect, in which the bodily sensation information is an extremity temperature of the person.

Accordingly, the air blown by the air blower can be controlled in accordance with the extremity temperature, which tends to influence the bodily sensations of the person.

Furthermore, for example, a sleep assistance system according to an eleventh aspect of the present disclosure is the sleep assistance system according to the tenth aspect, in which when the extremity temperature of the person is decreasing, the controller controls the air volume of the air blower to cause the air volume to decrease.

Since a person is more prone to feeling cold when their extremity temperature decreases at the same time that their core temperature is low, by controlling the air volume in the manner described above, it is possible to more readily facilitate the lowering of the core temperature in a manner such that the person does not feel cold.

Furthermore, for example, a sleep assistance system according to a twelfth aspect of the present disclosure is the sleep assistance system according to the ninth aspect, in which the bodily sensation information is a detection result of bodily movement of the person.

Accordingly, the air blown by the air blower can be controlled in accordance with the bodily movement of the person, which tends to change depending on the bodily sensations of the person.

Furthermore, for example, a sleep assistance system according to a thirteenth aspect of the present disclosure is the sleep assistance system according to the twelfth aspect, in which the person is sleeping in bedding, and the detection result is a detection result of bodily movement based on vibration of the bedding.

Accordingly, the accuracy of detection of bodily movements can be increased. Furthermore, since a bodily movement can be detected without having to attach a sensor to the person, it is possible to prevent such detection of bodily movements from causing a sleep disturbance for the person.

Furthermore, for example, a sleep assistance system according to a fourteenth aspect of the present disclosure is the sleep assistance system according to any one of the first aspect to the thirteenth aspect that further includes: a temperature adjuster that heats the air blown by the air blower.

Accordingly, it becomes possible to prevent the temperature of the extremity of the person from excessively decreasing.

Furthermore, for example, a sleep assistance system according to a fifteenth aspect of the present disclosure is the sleep assistance system according to the fourteenth aspect, in which the controller causes the temperature adjuster to heat the air blown by the air blower during a predetermined period of time until the person wakes up.

Accordingly, the air blown by the air blower is heated, during a period of time at a stage in which the core temperature increases as the person prepares to wake up, thereby making it possible to facilitate the raising of the core temperature of the person. As a result, the person can more comfortably wake up.

Furthermore, for example, a sleep assistance system according to a sixteenth aspect of the present disclosure is the sleep assistance system according to any one of the first aspect to the fifteenth aspect, in which the controller obtains a temperature of a sleeping environment of the person, and controls the air blown by the air blower based on the temperature of the sleeping environment obtained.

Accordingly, air that can reduce discomfort felt while sleeping can be blown at the extremity of the person in accordance with the temperature of the sleeping environment.

Furthermore, for example, a sleep assistance system according to a seventeenth aspect of the present disclosure is the sleep assistance system according to any one of the first aspect to the sixteenth aspect, in which the controller controls the air blown by the air blower based on season information.

Accordingly, air that can reduce discomfort felt while sleeping can be blown at the extremity of the person in accordance with the season.

Furthermore, for example, a sleep assistance system according to an eighteenth aspect of the present disclosure is the sleep assistance system according to any one of the first aspect to the seventeenth aspect, in which the controller obtains an extremity temperature of the person, and controls the air blown by the air blower based on the extremity temperature of the person obtained.

Accordingly, air that can reduce discomfort felt while sleeping can be blown at the extremity of the person in accordance with the extremity temperature of the person.

Furthermore, for example, a sleep assistance system according to a nineteenth aspect of the present disclosure is the sleep assistance system according to any one of the first aspect to the eighteenth aspect, in which the extremity is a foot of the person.

Accordingly, since air is blown at the foot of the person, which is a portion among the extremities of the person with a large surface area, the lowering of the core temperature of the person can be effectively facilitated.

Furthermore, for example, a sleep assistance system according to a twentieth aspect of the present disclosure is the sleep assistance system according to the nineteenth aspect, in which the person is sleeping in bedding, and the air blower blows air at the foot that is surrounded by the bedding.

Accordingly, the sleep assistance system becomes less prone to being influenced by the environment in the surroundings of the bedding.

Furthermore, for example, a sleep assistance system according to a twenty-first aspect of the present disclosure is the sleep assistance system according to the twentieth aspect, in which the bedding includes: under bedding that is disposed below the person; and over bedding that is disposed above the person, and a portion of the over bedding is fixed to the under bedding in a region in which the foot of the person is located.

Accordingly, even when the position of the foot changes due to the person rolling over while sleeping or the like, the space surrounded by the under bedding and the over bedding can be prevented from collapsing, and air can be blown at the foot in a stable manner by the air blower.

Furthermore, for example, a sleep assistance system according to a twenty-second aspect of the present disclosure is the sleep assistance system according to any one of the nineteenth aspect to the twenty-first aspect, in which when the person who is sleeping is viewed from above, the air blower blows air at the foot from a direction intersecting a head-to-toe height direction of the person.

Accordingly, even when the position of the foot changes due to the person rolling over while sleeping or the like, the foot becomes less prone to straying from the position at which the air is blown, and the air can be blown at the foot in a stable manner by the air blower.

Hereinafter, embodiments of the present disclosure will be described with reference to the drawings.

It should be noted that the embodiments described below merely illustrate general or specific examples of the present disclosure. The numerical values, shapes, materials, elements, the arrangement and connection of the elements, steps, the order of the steps, etc., described in the following embodiments are mere examples, and are therefore not intended to limit the present disclosure. Accordingly, among elements in the following embodiments, those not appearing in any of the independent claims will be described as optional elements.

It should be noted that the figures are schematic diagrams and are not necessarily precise illustrations. Moreover, elements that are substantially the same are given the same reference signs in the respective figures, and redundant descriptions may be omitted or simplified.

### Embodiment

### [Outline]

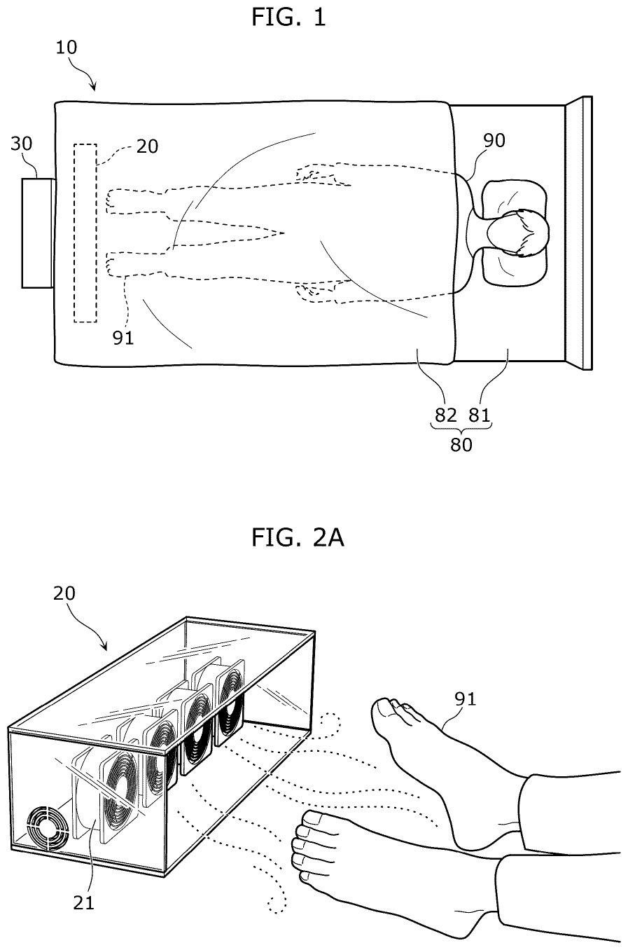

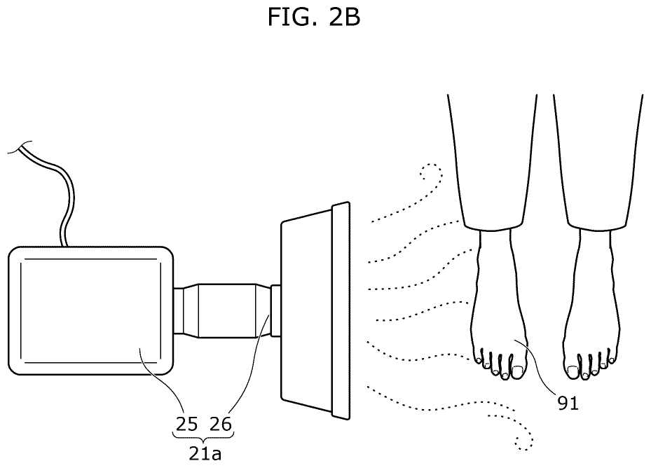

First, an outline of a sleep assistance system according to the present embodiment will be described with reference to FIG. 1 and FIG. 2A. FIG. 1 is a diagram of an outline for describing sleep assistance system 10 according to the embodiment. FIG. 2A is a diagram illustrating an inside of bedding 80 illustrated in FIG. 1. FIG. 1 is a plan view of bedding 80 in which person 90 is sleeping when viewed from above. Furthermore, in FIG. 1, dashed lines indicate outlines of person 90 and air blowing device 20 in bedding 80. Furthermore, FIG. 2A illustrates a vicinity of foot 91 of person 90 in which air blowing device 20 is disposed inside of bedding 80.

As illustrated in FIG. 1 and FIG. 2A, sleep assistance system 10 includes air blowing device 20 that includes air blower 21 and control device 30 that controls air blown by air blower 21.

Sleep assistance system 10 assists person 90 who is sleeping in bedding 80 or the like, by facilitating sleep. In the present specification, “sleeping” refers to a state in which person 90 has assumed a sleeping posture to sleep in bedding 80 or the like, and person 90 is considered to be sleeping as long as person 90 is trying to sleep even when person 90 has not actually fallen asleep. Bedding 80 includes, for example, under bedding 81, such as a mattress or a sleeping mat that is disposed below person 90, and over bedding 82, such as a blanket or a comforter that is disposed above person 90. In the example illustrated in FIG. 1 and FIG. 2A, foot 91 of person 90 is surrounded by bedding 80, and more specifically, is sandwiched between under bedding 81 and over bedding 82.

Furthermore, a portion of over bedding 82 in a region in bedding 80 in which foot 91 of person 90 is located, is fixed to under bedding 81, for example. The portion of over bedding 82 is, for example, fixed in place by being sandwiched between under bedding 81 and a floor, a bed frame, or the like that supports under bedding 81. The portion of over bedding 82 may be fixed to under bedding 81 using a fastener, such as a clip or the like. Accordingly, even when the position of foot 91 changes due to person 90 rolling over while sleeping or the like, the space surrounded by under bedding 81 and over bedding 82 can be prevented from collapsing, and air can be blown at foot 91 in a stable manner by air blower 21 that is described later. It should be noted that the region in bedding 80 in which foot 91 of person 90 is located is a region closer to the feet rather than the knees of person 90 in bedding 80.

Air blower 21 blows air at foot 91 that is an extremity of person 90 who is sleeping in bedding 80. Air blower 21 is disposed to directly blow air at foot 91 of person 90. Control device 30 (more specifically, controller 31 as described later) controls the air blown by air blower 21 to facilitate the lowering of a core temperature of person 90 who is sleeping in bedding 80. Control device 30 drives air blower 21 at an air volume at which the core temperature of person 90 can be significantly decreased, for example. According to sleep assistance system 10, the lowering of the core temperature of person 90 can be facilitated by simply blowing air at foot 91 of person 90, and the quality of sleep of person 90 can be enhanced in an efficient manner.

Is should be noted that an appearance of air blower 21 is not limited to the example illustrated in FIG. 2A, and is not particularly limited. FIG. 2B is another diagram illustrating the inside of bedding 80. FIG. 2B illustrates a plan view of person 90 who is sleeping in bedding 80 when viewed from above with bedding 80 omitted. Instead of air blower 21 as illustrated in FIG. 2A, air blowing device 20 may include air blower 21a as illustrated in FIG. 2B.

### [Configuration]

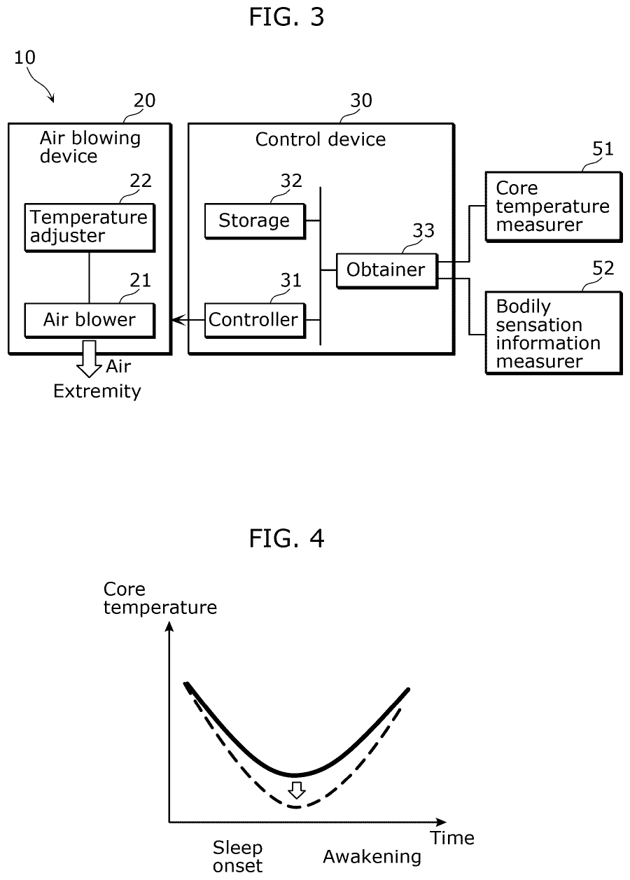

Hereinafter, a detailed configuration of sleep assistance system 10 will be described with reference to FIG. 3 in addition to FIG. 1, FIG. 2A, and FIG. 2B. FIG. 3 is a block diagram illustrating a configuration of sleep assistance system 10 according to the embodiment.

Sleep assistance system 10 includes air blowing device 20, control device 30, core temperature measurer 51, and bodily sensation information measurer 52.

Air blowing device 20 includes air blower 21 and temperature adjuster 22. Operation of air blowing device 20 is controlled by controller 31 of control device 30.

Air blower 21 is, for example, disposed between under bedding 81 and over bedding 82 at the feet of person 90. In the example illustrated in FIG. 2A, air blower 21 includes a plurality of air blowing units, such as fans. The plurality of air blowing units are arranged alongside the feet of person 90 in bedding 80 in a top view, for example. Furthermore, the plurality of air blowing units are surrounded by a frame in a direction other than a front-and-back direction of a direction in which the air is blown. In the example illustrated in FIG. 2A, the frame is transparent, but the frame may be opaque. The air blown by the air blowing units is blown to directly hit the sole of foot 91 of person 90, for example.

It should be noted that the configuration and arrangement of air blower 21 is not particularly limited as long as air blower 21 can blow air at foot 91 of person 90. For example, as described above, sleep assistance system 10 may include air blower 21a that is illustrated in FIG. 2B instead of air blower 21. Air blower 21a includes air blowing unit 25 and air duct 26, and air blown by air blowing unit 25 is blown at foot 91 of person 90 via air duct 26. In this case, an air outlet of air duct 26 is, for example, disposed between under bedding 81 and over bedding 82 at the feet of person 90. As long as a portion that includes the air outlet of air duct 26 is disposed between under bedding 81 and over bedding 82, other portions of air blower 21a may be disposed outside of bedding 80. Furthermore, in the example illustrated in FIG. 2B, the air outlet of air duct 26 spreads out to be wider than a portion of air duct 26 located closer to air blowing unit 25.

Furthermore, in the example illustrated in FIG. 2B, in a top view, when viewing person 90 who is sleeping from above, air blower 21a blows air at foot 91 from a direction intersecting a head-to-toe height direction (i.e., a direction that connects the head and the feet of person 90 when they are standing) of person 90. Accordingly, even when the position of foot 91 changes due to person 90 rolling over while sleeping or the like, foot 91 becomes less prone to straying from the position at which air is blown, and air can be blown at foot 91 in a stable manner by air blower 21a. Furthermore, the air outlet of air duct 26 can be miniaturized. The direction intersecting the head-to-toe height direction of person 90 may be a direction orthogonal to the head-to-toe height direction of person 90. Furthermore, as exemplified in air blower 21 illustrated in FIG. 2A, the air that exits air duct 26 may be blown to cause the air to directly hit the sole of foot 91 of person 90. Furthermore, in a top view, when viewing person 90 who is sleeping from above, air blower 21 may blow air at foot 91 from a direction intersecting the head-to-toe height direction of person 90. In sleep assistance system 10, since air blower 21 can be substituted with air blower 21a, it is possible to replace any mention of “air blower 21” with “air blower 21a” in the following description.

Furthermore, in air blower 21, the air volume can be changed, and the air volume can be adjusted by adjusting the electrical power supplied to air blower 21, for example. The air volume of air blower 21 may be changeable in a manner that is incremental, or may be changeable in a manner that is continuous.

Temperature adjuster 22 adjusts the temperature of the air blown by air blower 21. For example, temperature adjuster 22 includes heating wires or the like, and is a heater that heats the air blown by air blower 21. Accordingly, air blower 21 can blow warm air at foot 91 of person 90. For example, when controller 31 determines that a temperature of the surroundings of bedding 80 obtained from a temperature sensor or the like that is not illustrated in the figures is at or below a predetermined temperature, temperature adjuster 22 heats the air blown by air blower 21 based on control performed by controller 31. Accordingly, the temperature of foot 91 of person 90 is prevented from excessively decreasing. It should be noted that air blowing device 20 need not include temperature adjuster 22. Furthermore, temperature adjuster 22 may include a function to cool the air blown by air blower 21. For example, temperature adjuster 22 may be a thermoelectric element that is capable of both heating and cooling a Peltier device or the like.

Furthermore, temperature adjuster 22 may include a temperature sensor (not illustrated in the figures) that measures the air temperature of the air blown by air blower 21. A measurement result of the temperature sensor is used to control the air temperature of the air blown by air blower 21.

Control device 30 includes controller 31, storage 32, and obtainer 33.

Controller 31 is a processing circuit (processor) that controls operation of air blowing device 20. Controller 31 performs various information processing for controlling operation of air blowing device 20. Controller 31 controls the air blown by air blower 21. For example, controller 31 controls, based on the core temperature of person 90, the air volume of air blower 21. In the present specification, unless otherwise noted, the “air volume” of air blower 21 refers to an air volume per unit time of air blown by air blower 21. Furthermore, controller 31 may control the temperature of the air blown by air blower 21 (i.e., the air temperature) by controlling temperature adjuster 22. Control performed by controller 31 will be described in detail later. For example, controller 31 includes memory that stores a program or the like and a processor that executes the program. The program may be stored in storage 32.

Storage 32 stores information and data needed for processing performed by controller 31. Storage 32 is a non-volatile storage device, such as a semiconductor memory, for example. It should be noted that at least a portion of storage 32 is provided in a device that is separate from control device 30, and controller 31 may obtain data stored in storage 32 via a network, such as the Internet or the like.

Obtainer 33 communicates with core temperature measurer 51 and bodily sensation information measurer 52, and obtains data from core temperature measurer 51 and bodily sensation information measurer 52. Obtainer 33 includes a communication interface (communication circuit) for communicating with an external device or the like. Furthermore, obtainer 33 may include an input device that obtains input operation from a user. The input device is, for example, a touch panel, an operation switch, an operation button, or the like.

Core temperature measurer 51 measures the core temperature of person 90, and outputs a measurement result to control device 30. Core temperature measurer 51 is, for example, a core temperature measuring instrument that makes use of a heat flux compensation method or the like. Core temperature measurer 51 may be a device that measures one or more biometric information items of person 90, and calculates the core temperature based on the one or more biometric information items measured.

Bodily sensation information measurer 52 detects bodily sensation information that indicates bodily sensations of person 90, such as sensations of heat or cold. Bodily sensation information is, for example, information that indicates whether person 90 is feeling cold.

For example, bodily sensation information measurer 52 measures an extremity temperature of person 90 as bodily sensation information, and outputs a measurement result to control device 30. In this case, bodily sensation information measurer 52 is an extremity temperature measuring instrument that measures the extremity temperature of person 90. Specifically, bodily sensation information measurer 52 measures a surface temperature of foot 91 of person 90 (i.e., skin temperature of foot 91) as an extremity temperature. The extremity temperature measuring instrument may be an instrument that uses a contact temperature sensor, or may be an instrument that uses a non-contact temperature sensor.

It should be noted that sleep assistance system 10 need not include at least one of core temperature measurer 51 or bodily sensation information measurer 52, and obtainer 33 may obtain at least one of the core temperature of person 90 or bodily sensation information by communicating with one or more external devices that include a function of at least one of core temperature measurer 51 or bodily sensation information measurer 52. Furthermore, in this case, the external device may be a dedicated measuring instrument, or may be an information terminal, a wearable terminal, or the like that is possessed by the user.

### [Core Temperature Measurement Results]

Next, measurement results obtained by the inventors will be described that indicate how core temperatures change due to air being blown at the feet of test subjects that are sleeping in bedding.

Specifically, the inventors measured the core temperature of four Japanese male test subjects (age: 23.0±1.9 years, height: 169.6±4.5 cm, weight: 58.1±2.7 kg) under condition 1 to condition 3 as described below.
Condition 1: Air was not blown at the feet of the test subjects.
Condition 2: Through the entire night, each single fan blew air at an air volume of 0.9 m³/min at the feet of the test subjects.
Condition 3: Over a period of time starting from when the test subjects go to bed until three hours later, each single fan blew air at an air volume of 1.4 m³/min at the feet of the test subjects, and afterwards, each single fan blew air at an air volume of 0.9 m³/min.

Under each of these condition, the room temperature was controlled to keep the room temperature at 26° C., and the air blown by the fans was not heated. Furthermore, four fans were arranged at the feet of each of the test subjects inside the bedding.

The core temperatures were measured at 30-second intervals using a heat flux compensation-type core temperature gauge (CM-210 made by Terumo), and median values of the measurement results obtained while the test subjects were sleeping were compared as representative values of the core temperatures obtained while the test subjects were sleeping. A decrease in a median value of a core temperature indicates a decrease measured in overall core temperature while the test subject is sleeping.

TABLE 1 shows the median values of the measurement results of the core temperatures of test subject 1 to test subject 4 who are the four test subjects. Furthermore, in TABLE 1, determination results that indicate whether core temperatures are significantly decreasing, when comparing “Condition 1” with “Condition 2” and “Condition 3”, are shown in the columns titled “Significant difference”. In the columns titled “Significant difference”, a circle indicates that there is a significant decrease in core temperature when compared to “Condition 1”. It should be noted that determination was performed based on the Mann-Whitney U test, and a circle is indicated in cases where the p-value is less than 0.05. Furthermore, an interquartile range was used to exclude outlying values. In the columns titled “Significant difference”, a hyphen indicates a case where it was determined that the conditions indicated by a circle were not satisfied, and that there is no significant decrease in core temperature when compared to “Condition 1”.

TABLE 1

| | Condition 1 | Condition 2 | | Condition 3 | |
|---|---|---|---|---|---|
| | Core | Core | | Core | |
| | temper- | temper- | | temper- | |
| Test | ature | ature | Significant | ature | Significant |
| subject | [° C.] | [° C.] | difference | [° C.] | difference |
| Test | 36.33 | 35.70 | ◯ | 35.77 | ◯ |
| subject 1 | | | | | |
| Test | 35.68 | 35.42 | ◯ | 34.705 | ◯ |
| subject 2 | | | | | |
| Test | 35.93 | 35.22 | ◯ | 35.71 | ◯ |
| subject 3 | | | | | |
| Test | 35.61 | 35.48 | — | 35.11 | ◯ |
| subject 4 | | | | | |

As illustrated in TABLE 1, in “Condition 2”, in which through the entire night, each single fan blew air at an air volume of 0.9 m³/min at the feet of the test subjects, a significant decrease in core temperature was identified for three out the four test subjects. Furthermore, in “Condition 3”, in which over a period of time starting from when the test subjects go to bed until three hours later, the air volume was increased to be larger than that of “Condition 2”, a significant decrease in core temperature was identified for all of the four test subjects. It should be noted that in “Condition 2” and “Condition 3”, in cases where it was determined that core temperatures had significantly decreased, p-values were smaller than 0.01.

Based on the above results, it was discovered that it is possible to significantly decrease the core temperature of a person by controlling the air volume of the air blown at a foot of the person. Furthermore, it was also discovered that the core temperature of a person can be effectively lowered by increasing the air volume during a predetermined period of time starting from when the person goes to bed.

Accordingly, in sleep assistance system 10, controller 31 can facilitate the lowering of a core temperature of person 90 who is sleeping in bedding 80 by properly controlling the air volume of air blower 21. For example, in storage 32, an air volume of air blower 21 that can cause the core temperature of person 90 who is sleeping in bedding 80 to significantly decrease, which is established in advance by experiments and the like, is stored, and controller 31 controls the air volume of air blower 21 to cause the air volume to match the air volume stored in storage 32. Furthermore, controller 31 may control an air volume of air blower 21 to cause the air volume of air blower 21 during a predetermined period of time starting from when person 90 goes to bed to be larger than the air volume of air blower 21 when person 90 wakes up.

FIG. 4 is a drawing schematically illustrating change in a core temperature when air is blown at foot 91 of person 90 who is sleeping in bedding 80. In FIG. 4, the solid line indicates the change in core temperature of person 90 in a case in which air is not blown at foot 91 of person 90, and the dashed line indicates the change in core temperature of person 90 in a case in which air is properly blown at foot 91 of person 90. As illustrated in FIG. 4, broadly speaking, the core temperature of person 90 who is sleeping decreases up to a given point in time after person 90 goes to bed, and subsequently, begins increasing and continues to increase until person 90 wakes up. Furthermore, it is known that the lower the core temperature of the person sleeping is, the more the quality of sleep increases. Accordingly, the quality of sleep of person 90 can be increased by blowing air at a foot of person 90 at an air volume that facilitates the lowering of the core temperature of person 90 who is sleeping.

In this manner, according to sleep assistance system 10, the lowering of the core temperature of person 90 who is sleeping can be facilitated by simply blowing air at foot 91 of person 90, and the quality of sleep of person 90 can be enhanced. For example, in sleep assistance system 10, environmental factors, such as temperature and humidity in the bed need not be controlled by using a plurality of hygrothermal sensors, as is described in PTL 1. Accordingly, in sleep assistance system 10, the quality of sleep of person 90 can be enhanced in an efficient manner. Furthermore, in sleep assistance system 10, since a wide range of environmental factors in the bed need not be controlled, it is possible to conserve energy.

### [Operation]

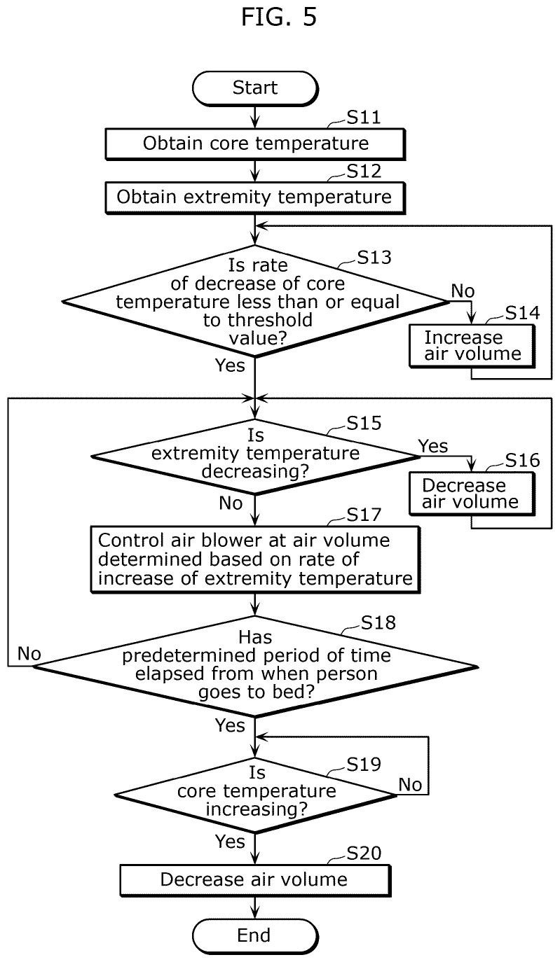

Next, an example of operation of sleep assistance system 10 according to the embodiment will be described. FIG. 5 is a flowchart illustrating an example of operation of sleep assistance system 10 according to the embodiment.

First, person 90 (i.e., a user of sleep assistance system 10) that is trying to sleep enters bedding 80, and by the actuation of air blower 21 being turned on or the like, air blower 21 starts blowing air at a predetermined air volume at foot 91 of person 90. The predetermined air volume is, for example, an air volume that can facilitate the lowering of a core temperature of a typical person who is sleeping, which is established in advance by experiments and the like. Furthermore, the predetermined air volume may be set to a value that corresponds to the user. Then, controller 31 obtains, via obtainer 33, the core temperature of person 90 who is sleeping in bedding 80 that is measured by core temperature measurer 51 (step S11). For example, core temperature measurer 51 measures the core temperature of person 90 at a predetermined interval. Controller 31 causes a result of the core temperature measured at the predetermined interval to be stored in storage 32 as a core temperature history together with time information. The obtaining of the core temperature of person 90 performed by controller 31 is continued at the predetermined interval until person 90 wakes up. The predetermined interval is, for example, no less than 10 seconds and no more than 10 minutes.

Furthermore, controller 31 obtains, via obtainer 33, an extremity temperature (more specifically, a temperature of foot 91), as bodily sensation information, of person 90 who is sleeping in bedding 80 that is measured by bodily sensation information measurer 52 (step S12). For example, bodily sensation information measurer 52 measures the extremity temperature of person 90 at the predetermined interval, and controller 31 causes a result of the extremity temperature measured at the predetermined interval to be stored in storage 32 as an extremity temperature history together with time information. The obtaining of the extremity temperature of person 90 performed by controller 31 is continued at the predetermined interval until person 90 wakes up. The predetermined interval is, for example, no less than 10 seconds and no more than 10 minutes.

Next, after a given period of time has elapsed from when person 90 goes to bed, controller 31 determines whether a rate of decrease of the core temperature of person 90 is less than or equal to a predetermined threshold value (step S13). For example, controller 31 refers to the core temperature history of person 90 who is sleeping in bedding 80 that is stored in storage 32, and determines whether the rate of decrease of the core temperature is less than or equal to the predetermined threshold value. The rate of decrease of the core temperature used for this determination is, for example, an average speed that is calculated from an amount of change of the core temperature over a given period of time immediately before the determination is performed. Furthermore, the predetermined threshold value used for this determination is, for example, set to a value that is larger than the rate of decrease of the core temperature of a typical person who is sleeping. Furthermore, the rate of decrease of the core temperature of person 90 who is sleeping in a state in which sleep assistance system 10 is not being used may be measured in advance, and the predetermined threshold value may be set to a value that is larger than the rate of decrease measured.

Furthermore, for example, the point in time at which person 90 goes to bed is the point in time at which air blower 21 starts blowing air at a predetermined air volume at foot 91 of person 90, however, this example is not limiting, and the point in time may be set as appropriate. For example, the point in time may be set in advance to a time at which person 90 goes to bed, and may be set to a point in time at which a sensor or the like not illustrated in the figures detects that person 90 has entered bedding 80. Furthermore, the given period of time is, for example, no less than 10 minutes and no more than 30 minutes.

When it is determined that the rate of decrease of the core temperature of person 90 is not less than or equal to the predetermined threshold value (“No” in step S13), controller 31 then controls air blower 21 to increase the air volume of air blower 21 (step S14). At this point, although the air volume of air blower 21 may be increased by an amount of increase that is a value that has been determined in advance, controller 31 may determine the amount of increase by which the air volume is increased based on the rate of decrease of the core temperature of person 90. For example, the larger the difference between the rate of decrease of the core temperature of person 90 and the predetermined threshold value, the larger the amount of increase of the air volume of air blower 21 is. Then, after a given period of time has elapsed, step S13 is once again performed. The given period of time is, for example, no less than 10 minutes and no more than 30 minutes.

Furthermore, in step S14, controller 31 may cool the air blown by air blower 21 by using temperature adjuster 22 in addition to or in lieu of control performed to increase the air volume of air blower 21. Furthermore, when two or more iterations of step S14 are performed, such cooling performed by temperature adjuster 22 may be performed to coincide with a predetermined iteration of step S14 for the second time step S14 is performed or onward. Accordingly, even for a condition where it is difficult for the core temperature to decrease due to the temperature in the surroundings of bedding 80 being high or the like, the lowering of the core temperature can be facilitated without substantially increasing the air volume.

In this manner, by performing step S13 and step S14, as needed, controller 31 can control the air blown by air blower 21 to cause the rate of decrease of the core temperature of person 90 who is sleeping in bedding 80 to be greater than or equal to the predetermined threshold value.

That being said, when it is determined that the rate of decrease of the core temperature of person 90 is less than or equal to the predetermined threshold value (“Yes” in step S13), controller 31 then determines whether the extremity temperature of person 90 is decreasing (step S15). For example, controller 31 refers to the extremity temperature history of person 90 who is sleeping in bedding 80 that is stored in storage 32, and determines whether the extremity temperature is trending toward decreasing over a given period of time immediately before determination is performed. It should be noted that in step S15, controller 31 may determine whether the extremity temperature of person 90 is decreasing and whether the rate of decrease of the extremity temperature is greater than or equal to a predetermined threshold value.

When it is determined that the extremity temperature of person 90 is decreasing (“Yes” in step S15), controller 31 then controls air blower 21 to decrease the air volume of air blower 21 (step S16). At this point, although the air volume of air blower 21 may be decreased by an amount of decrease that is a value that has been determined in advance, controller 31 may determine the amount of decrease by which the air volume of air blower 21 is decreased based on the rate of decrease of the extremity temperature of person 90. For example, the larger the rate of decrease of the extremity temperature of person 90, the larger the amount of decrease of the air volume of air blower 21 is. Then, after a given period of time has elapsed, step S15 is once again performed. The given period of time is, for example, no less than 10 minutes and no more than 30 minutes. It should be noted that after step S16, step S13 may once again be performed instead of step S15.

Furthermore, in step S16, controller 31 may cause the air blown by air blower 21 to be heated by using temperature adjuster 22 in addition to or in lieu of control performed to decrease the air volume of air blower 21. Furthermore, when two or more iterations of step S16 are performed, such heating performed by temperature adjuster 22 may be performed to coincide with a predetermined iteration of step S16 for the second time step S16 is performed or onward. Accordingly, even in a case in which a condition tends to cause the extremity temperature to decrease due to the temperature in the surroundings of bedding 80 being low or the like, the lowering of the extremity temperature can be inhibited without substantially decreasing the air volume.

In this manner, by performing step S15, and if necessary, by performing step S16, controller 31 can control air blown by air blower 21 to prevent the extremity temperature of person 90 who is sleeping from decreasing.

On the other hand, when it is determined that the extremity temperature of person 90 is not decreasing (“No” in step S15), controller 31 then controls air blower 21 at an air volume that is based on a rate of increase of the extremity temperature of person 90 (step S17). For example, when the rate of increase of the extremity temperature of person 90 is no less than zero and less than or equal to a predetermined value, controller 31 determines not to change the current air volume of air blower 21. Furthermore, for example, when the rate of increase of the extremity temperature of person 90 is larger than the predetermined value, controller 31 determines the air volume such that the larger the difference between the predetermined value and the rate of increase of the extremity temperature of person 90, the larger the air volume becomes relative to the current air volume of air blower 21. Furthermore, when the rate of increase of the extremity temperature of person 90 is larger than the predetermined value, controller 31 may cause the current air volume of air blower 21 to increase by a predetermined amount. Furthermore, the predetermined value may be zero.

After step S17, controller 31 determines whether a predetermined period of time has elapsed from the point in time when person 90 goes to bed (step S18). The length of the predetermined period of time is, for example, no less than two hours and no more than five hours. When it is determined that the predetermined period of time has not elapsed from the point in time when person 90 goes to bed (“No” in step S18), controller 31 once again performs step S15 after a given period of time has elapsed. The given period of time is, for example, no less than one minute and no more than 30 minutes. Here, it should be noted that step S13 may once again be performed instead of step S15. Furthermore, in this case, step S18 may once again be performed instead of step S15, and the current air volume determined in step S17 may be maintained until a predetermined period of time has elapsed.

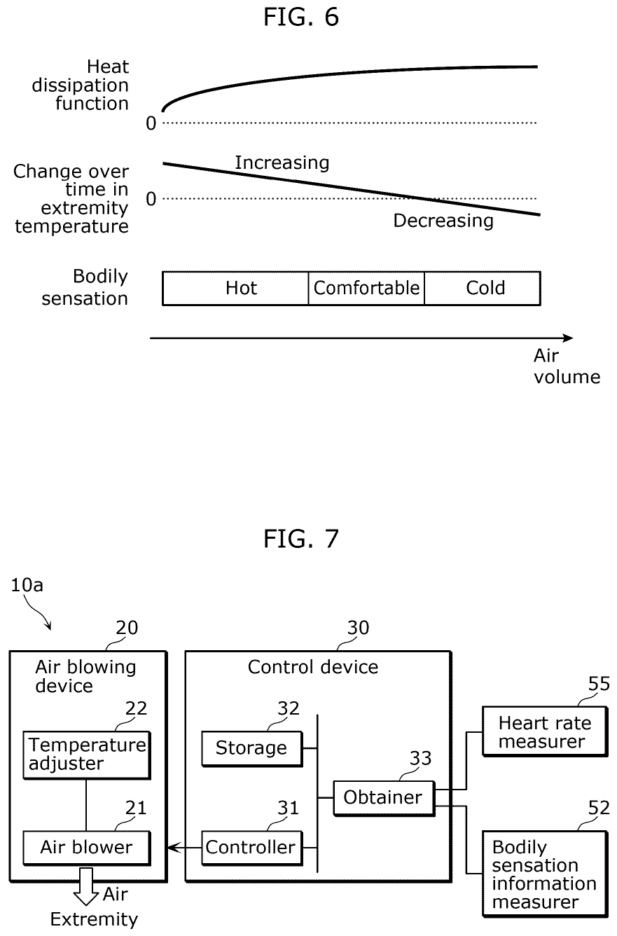

In this manner, by performing step S13 through step S17, during a predetermined period of time starting from when person 90 goes to bed, controller 31 can control the air volume of air blower 21 to maximize the air volume of air blower 21 to an extent that the extremity temperature of person 90 who is sleeping in bedding 80 does not decrease. Accordingly, since the lowering of the core temperature of person 90 can be facilitated while preventing person 90 from feeling cold, the quality of sleep of person 90 can be further enhanced. This point will be described in detail with reference to FIG. 6. FIG. 6 is a drawing schematically illustrating a relationship between an air volume of air blower 21, a heat dissipation function of person 90, a rate of change of an extremity temperature, and a bodily sensation when person 90 is sleeping. FIG. 6 illustrates how the heat dissipation function of person 90, the rate of change of the extremity temperature, and the bodily sensation change in response to increases in the air volume of air blower 21. For descriptive purposes, FIG. 6 merely indicates a simplified illustration of how the heat dissipation function of person 90, the rate of change of the extremity temperature, and the bodily sensation change, and, in reality, since the environment and individual differences and the like can have an influence, FIG. 6 is not necessarily illustrative of this. For example, change over time of the extremity temperature does not necessarily change in a linear manner in accordance with the air volume. Furthermore, for example, the air volume levels at the boundaries of each bodily sensation may also vary.

As illustrated in FIG. 6, when the air volume of air blower 21 changes, the heat dissipation function of person 90, the rate of change of the extremity temperature, and the bodily sensation change in response to the changes in the air volume of air blower 21. Specifically, the larger the air volume of air blower 21 is, the larger the heat dissipation function of person 90 becomes. As a result, the core temperature of person 90 decreases due to heat dissipation, and the larger the air volume of air blower 21 is, the larger the rate of decrease of the core temperature of person 90 becomes. On the other hand, since the extremity temperature of person 90 who is sleeping increases as heat in the core of person 90 moves toward their extremities in order to cause the core temperature to decrease, when air is not blown at the extremities, the rate of increase becomes large. The larger the air volume of air blower 21 is, the less prone to increasing the extremity temperature of person 90 who is sleeping becomes, and at a predetermined air volume, the extremity temperature of person 90 begins decreasing. Furthermore, person 90 is more prone to feeling hot when the rate of increase of the extremity temperature becomes high, and person 90 is more prone to feeling comfortable when the extremity temperature is at or below a given level. Additionally, when the extremity temperature decreases in a state in which the core temperature is low, person 90 is more prone to feeling cold. Accordingly, by controller 31 maximizing the air volume of air blower 21 to an extent that the rate of change of the extremity temperature of person 90 who is sleeping satisfies a predetermined condition, or for example, by maximizing the air volume of air blower 21 to an extent that the extremity temperature does not decrease, it is thereby possible to maximize the heat dissipation function while preventing person 90 from feeling cold. As a result, the lowering of the core temperature of person 90 is effectively facilitated, thereby making it possible to further enhance the quality of sleep of person 90.

Referring again to FIG. 5, when it is determined that a predetermined period of time has elapsed from the point in time when person 90 goes to bed (“Yes” in step S18), controller 31 then determines whether the core temperature of person 90 who is sleeping is increasing (step S19). For example, controller 31 refers to the core temperature history of person 90 who is sleeping in bedding 80 that is stored in storage 32, and determines whether the core temperature is trending toward increasing over a given period of time.

When it is determined that the core temperature of person 90 who is sleeping is not increasing (“No” in step S19), controller 31 maintains the current air volume of air blower 21, and step S19 is once again performed after a given period of time has elapsed. On the other hand, when it is determined that the core temperature of person 90 who is sleeping is increasing (“Yes” in step S19), controller 31 then controls the air volume to decrease the air volume of air blower 21 (step S20). Subsequently, air continues to be blown until person 90 wakes up and the actuation of air blower 21 is turned off. For example, controller 31 controls the air volume of air blower 21 to cause the air volume to be less than the air volume at the point in time at which air blower 21 starts blowing air. Furthermore, controller 31 may reduce the air volume of air blower 21 down to zero. In other words, controller 31 may stop actuation of air blower 21, and end operation of sleep assistance system 10 in step S20.

Furthermore, in step S20, controller 31 may cause temperature adjuster 22 to heat the air blown by air blower 21 in lieu of or in addition to control performed to decrease the air volume of air blower 21. In other words, controller 31 may cause temperature adjuster 22 to heat the air blown by air blower 21 over a predetermined period of time until person 90 wakes up. Accordingly, the air blown by air blower 21 is heated, over a period of time at a stage in which the core temperature increases as person 90 prepares to wake up, as was described with reference to FIG. 4, thereby making it possible to facilitate raising of the core temperature of person 90. As a result, person 90 can more comfortably wake up.

In this manner, during a predetermined period of time starting from when person 90 goes to bed, controller 31 controls the air volume of air blower 21 to facilitate the lowering of the core temperature of person 90 who is sleeping, and after the predetermined period of time is over, controller 31 performs control to decrease the air volume of air blower 21. In other words, controller 31 controls the air volume of air blower 21 to cause the air volume of air blower 21 during a predetermined period of time starting from when person 90 goes to bed to be larger than the air volume of air blower 21 when person 90 wakes up. Accordingly, since this facilitates the lowering of the core temperature of person 90, during a predetermined period of time starting from when person 90 goes to bed, which is a period that greatly influences the quality of sleep of person 90, the quality of sleep of person 90 can be effectively enhanced. In this case, the predetermined period of time may be determined in advance as described above, or the predetermined period of time may be determined by controller 31 as a period of time starting from when the core temperature of person 90 who is sleeping begins decreasing until a given point in time at which the core temperature of person 90 who is sleeping begins increasing, or a period of time based on the given point in time.

It should be noted that as long as sleep assistance system 10 operates to control the air blown by air blower 21 to facilitate the lowering of the core temperature of person 90 who is sleeping, at least a portion of the above-mentioned operations may be omitted. For example, step S15 to step S17 may be omitted, at least one of step S18 or step S19 may be omitted, and step S18 to step S20 may be omitted.

Furthermore, in step S15, controller 31 may obtain, as bodily sensation information, a detection result of bodily movement of person 90 who is sleeping instead of the extremity temperature of person 90 who is sleeping. In other words, in this case, the bodily sensation information is a detection result of bodily movement of person 90 who is sleeping in bedding 80. Moreover, in step S15, controller 31 determines whether an amount of bodily movement of person 90 who is sleeping is greater than or equal to a predetermined amount. When controller 31 determines that the amount of bodily movement of person 90 who is sleeping is greater than or equal to the predetermined amount, the procedure then proceeds to step S16. Furthermore, when controller 31 determines that the amount of bodily movement of person 90 who is sleeping is not greater than or equal to the predetermined amount, in step S17, controller 31 then controls the air volume of air blower 21 at the air volume determined based on the amount of bodily movement of person 90 who is sleeping. Accordingly, the air volume of air blower 21 is controlled in accordance with bodily movements of person 90, which tend to change depending on bodily sensations of person 90. Furthermore, in this case, in lieu of or in addition to the extremity temperature measuring instrument, bodily sensation information measurer 52 includes a bodily movement detector that includes an acceleration sensor that is attached to person 90, a plurality of pressure sensors attached to bedding 80, an image sensor that captures an image of person 90, or the like, and bodily sensation information measurer 52 outputs a detection result of such sensors as a detection result of bodily movement.

Furthermore, the bodily movement detector included in bodily sensation information measurer 52 may include a sensor that detects vibration of bedding 80, such as an acceleration sensor that is attached to bedding 80. In this case, the detection result of bodily movement is a result of bodily movement detected based on vibration of bedding 80 that is detected by the sensor. Accordingly, the accuracy of detection of bodily movements can be increased. Furthermore, since a sensor can be easily attached to bedding 80 without the need to attach a sensor to person 90, with a simple configuration, it is thereby possible to prevent such detection of bodily movements from causing a sleep disturbance for person 90.

### [Variation 1]

Next, Variation 1 of the embodiment will be described. Hereinafter, in the description of Variation 1, the description will focus on the points of difference with the embodiment, and overlapping description will be omitted or simplified.

FIG. 7 is a block diagram illustrating a configuration of sleep assistance system 10a according to the present variation. As illustrated in FIG. 7, when compared to sleep assistance system 10 according to the embodiment, sleep assistance system 10a according to the present variation differs by including heart rate measurer 55 instead of core temperature measurer 51.

Heart rate measurer 55 measures a heart rate of person 90, and outputs a measurement result to control device 30. Heart rate measurer 55 is a heart rate measuring instrument.

It should be noted that sleep assistance system 10a need not include heart rate measurer 55, and may obtain the heart rate of person 90 by obtainer 33 communicating with an external device that includes a function of heart rate measurer 55. Furthermore, in this case, the external device may be a dedicated measuring instrument, or may be a wearable terminal or the like that is possessed by the user.

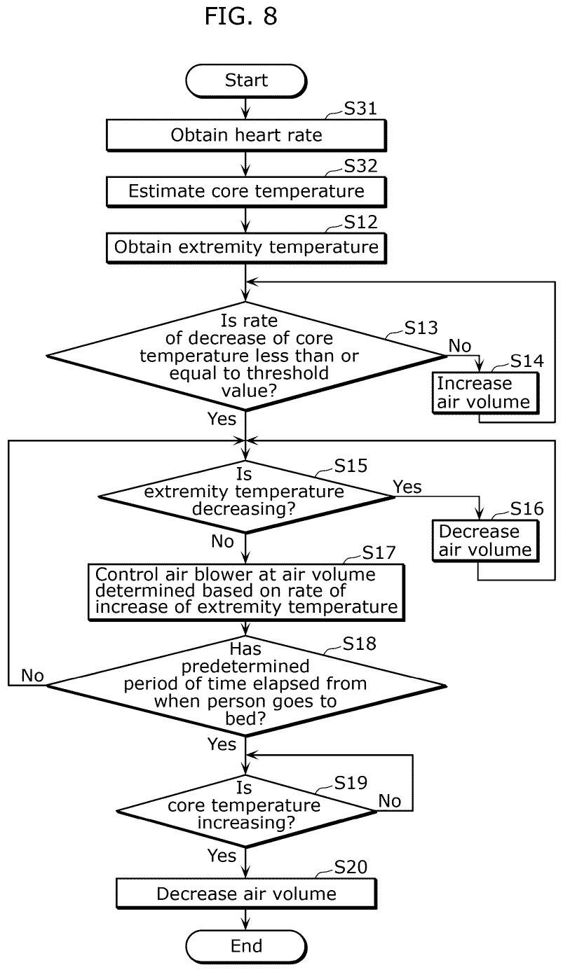

Next, an example of operation of sleep assistance system 10a according to the present variation will be described. FIG. 8 is a flowchart illustrating an example of operation of sleep assistance system 10a according to the present variation.

First, person 90 (i.e., a user of sleep assistance system 10a) that is trying to sleep enters bedding 80, and by the actuation of air blower 21 being turned on or the like, air blower 21 starts blowing air at a predetermined air volume at foot 91 of person 90. Then, controller 31 obtains, via obtainer 33, a heart rate of person 90 who is sleeping in bedding 80 that is measured by heart rate measurer 55 (step S31). For example, heart rate measurer 55 measures the heart rate of person 90 at a predetermined interval. The obtaining of the heart rate of person 90 performed by controller 31 is continued at the predetermined interval until person 90 wakes up. The predetermined interval is, for example, no less than 10 seconds and no more than 10 minutes.

Next, controller 31 estimates the core temperature of person 90 who is sleeping in bedding 80 based on the heart rate obtained (step S32). Controller 31 causes the core temperature estimated from the heart rate measured at the predetermined interval to be stored in storage 32 as a core temperature history together with time information. Core temperatures have a tendency to become higher in relation to how high the heart rate is, and controller 31 estimates the core temperature from the heart rate based on a function, a data table, or the like that represents the relationship between heart rates and core temperatures, for example.

After step S32, the same operations as described for step S12 and onward, which were described as the operations for the above-mentioned sleep assistance system 10, are performed.

Since sleep assistance system 10a does not directly measure the core temperature of person 90, and instead measures the heart rate, which can be more easily measured than the core temperature, and estimates the core temperature from the heart rate measured, this makes it possible to more easily obtain information on the core temperature.

### [Variation 2]

Next, Variation 2 of the embodiment will be described. Hereinafter, in the description of Variation 2, the description will focus on the points of difference with the embodiment and Variation 1 of the embodiment, and overlapping description will be omitted or simplified.

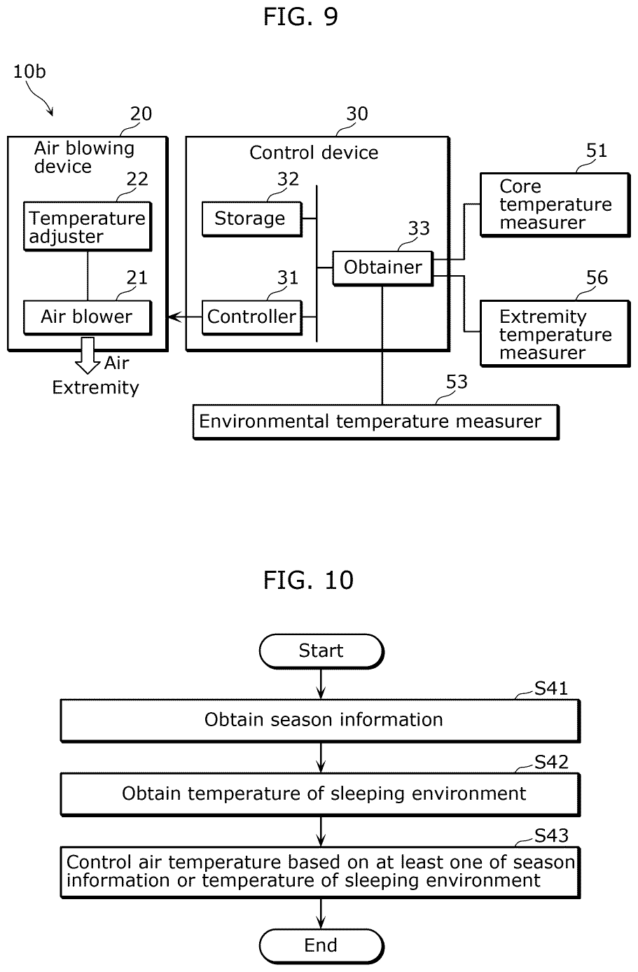

FIG. 9 is a block diagram illustrating a configuration of sleep assistance system 10b according to the present variation. As illustrated in FIG. 9, when compared to sleep assistance system 10 according to the embodiment, sleep assistance system 10b according to the present variation differs by further including environmental temperature measurer 53, and by including extremity temperature measurer 56 instead of bodily sensation information measurer 52.

In sleep assistance system 10b, controller 31 controls the air temperature of air blown by air blower 21 by using temperature adjuster 22 based on the information obtained.

Environmental temperature measurer 53 measures a temperature of the sleeping environment of person 90. The temperature of the sleeping environment of person 90 is, for example, a temperature of the surroundings of bedding 80 in which person 90 sleeps, and a specific example is a room temperature of a room in which bedding 80 is disposed. Environmental temperature measurer 53 outputs a measurement result of the temperature of the sleeping environment of person 90 to control device 30. Obtainer 33 of control device 30 obtains the measurement result from environmental temperature measurer 53. Environmental temperature measurer 53 is a temperature sensor that measures a temperature, and is, for example, provided in the room in which bedding 80 is disposed.

Extremity temperature measurer 56 is an extremity temperature measuring instrument that measures an extremity temperature of person 90. Extremity temperature measurer 56 measures a surface temperature of foot 91 of person 90 (i.e., skin temperature of foot 91) as an extremity temperature. The extremity temperature measuring instrument may be an instrument that uses a contact temperature sensor, or may be an instrument that uses a non-contact temperature sensor. Furthermore, the extremity temperature measured by extremity temperature measurer 56 may be used as the above-mentioned bodily sensation information.

It should be noted that sleep assistance system 10b need not include at least one of environmental temperature measurer 53 or extremity temperature measurer 56, and obtainer 33 may obtain at least one of the temperature of the sleeping environment of person 90 or the extremity temperature by communicating with one or more external devices that include a function of at least one of environmental temperature measurer 53 or extremity temperature measurer 56. Furthermore, in this case, the external device may be a dedicated measuring instrument, or may be an information terminal, a wearable terminal, or the like that is possessed by the user. Furthermore, sleep assistance system 10b may further include the bodily movement detector that is used by bodily sensation information measurer 52 as described in the above-mentioned configuration of sleep assistance system 10. Furthermore, instead of core temperature measurer 51, sleep assistance system 10b may include heart rate measurer 55, which was described as an element of the above-mentioned sleep assistance system 10a.

Next, an example of operation of sleep assistance system 10b according to the present variation will be described. FIG. 10 is a flowchart illustrating an example of operation of sleep assistance system 10b according to the present variation. In the example of operation illustrated in FIG. 10, controller 31 controls the air temperature of air blown by air blower 21 by using temperature adjuster 22. It should be noted that controller 31 may perform the operations illustrated in FIG. 10 in parallel with the operations described with reference to FIG. 5. In other words, controller 31 may control both the air temperature of air blown by air blower 21 and an air volume.

First, person 90 (i.e., a user of sleep assistance system 10b) that is trying to sleep enters bedding 80, and by the actuation of air blower 21 being turned on or the like, air blower 21 starts blowing air at a predetermined air volume at foot 91 of person 90. Then, controller 31 obtains season information that indicates the current season (step S41). For example, controller 31 includes a timing circuit, and determines the current date by measuring time, and obtains the date determined as season information. Furthermore, controller 31 may obtain the current date and the like as season information via obtainer 33 from an external device using a network, such as the Internet. Furthermore, controller 31 may obtain season information that is input by a user via obtainer 33. The season information may be information that indicates the current month or date, and may be information that indicates which of the four seasons the current season is.

Next, controller 31 obtains, via obtainer 33, the temperature of the sleeping environment of person 90 that is measured by environmental temperature measurer 53 (step S42).

Next, controller 31 controls the air temperature of the air blown by air blower 21 based on at least one of the season information or the temperature of the sleeping environment obtained (step S43). Controller 31 determines the air temperature of the air blown by air blower 21 based on at least one of the season information or the temperature of the sleeping environment, and, by using temperature adjuster 22, adjusts the air temperature of the air blown by air blower 21 to match the air temperature determined. For example, storage 32 stores information indicating a corresponding relationship of a table or the like in which at least one of the season information or the temperature of the sleeping environment is associated with the air temperature, and controller 31 determines the air temperature by referring to the information. Although the temperature at which person 90 feels comfortable may change depending on the season and the temperature of the sleeping environment, by controlling the air temperature, the likelihood of causing discomfort for person 90 during sleep is reduced.

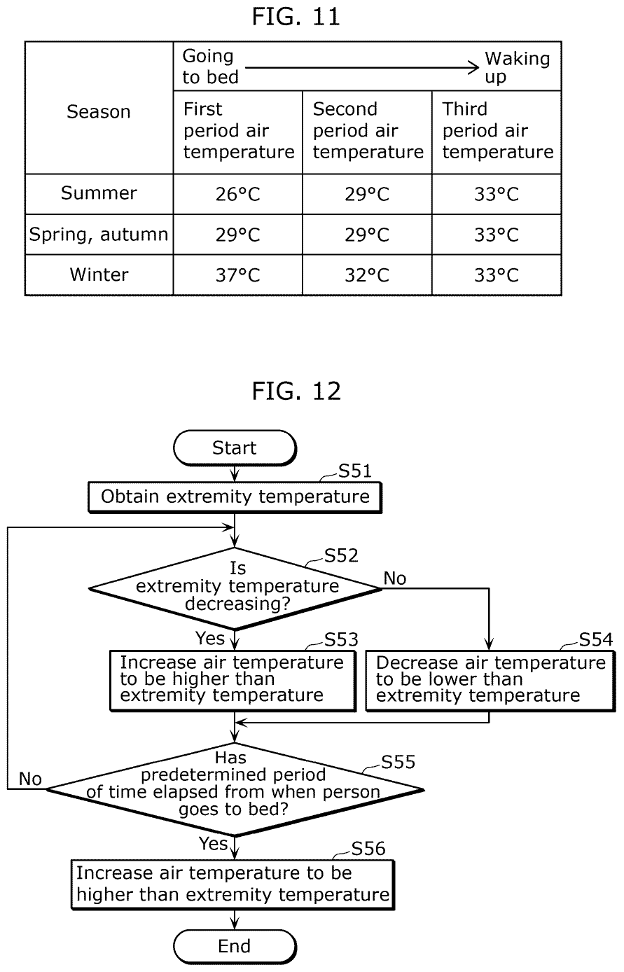

FIG. 11 is a diagram for describing an example of air temperature control performed by controller 31. In FIG. 11, an example of air temperatures is illustrated in a case in which controller 31 controls the air temperature of the air blown by air blower 21 based on season information. It should be noted that the example of air temperatures illustrated in FIG. 11 is merely an example, and air temperatures are not limited to those shown in the example in FIG. 11.

As illustrated in FIG. 11, controller 31 determines the air temperature of air blown by air blower 21 based on the season indicated by the season information. For example, controller 31 determines the air temperature such that, the lower the temperature of the season, the higher the air temperature is set to. Accordingly, since the air temperature changes depending on the season, the lowering of the core temperature is facilitated by the air blown by air blower 21, while making it easier for person 90 to comfortably fall asleep without causing discomfort.

Furthermore, as illustrated in FIG. 11, controller 31 may change the air temperature over a plurality of periods of time by which the period of time starting from when person 90 goes to bed until when person 90 wakes up is divided. In the example shown in FIG. 11, the period of time from the point in time when person 90 goes to bed until when person 90 wakes up is divided into a first period, a second period, and a third period, and controller 31 determines the air temperature in accordance with which period of time it is. For example, controller 31 determines the air temperature to be a different air temperature for the first period, which is a predetermined period of time starting from when person 90 goes to bed, and determines a different air temperature for the third period, which is a predetermined period of time until when person 90 wakes up. Furthermore, controller 31 may determine the air temperature to be a different air temperature for the second period, which is a period of time between the first period and the third period, and may determine a different air temperature for at least one of the first period or the third period.

Furthermore, for example, controller 31 determines the air temperature such that the air temperature for the third period is higher than the air temperature for at least one of the first period or the second period. Accordingly, the air temperature of air blown by air blower 21 increases, over a period of time at a stage in which the core temperature increases as person 90 prepares to wake up, as was described with reference to FIG. 4, thereby making it possible to facilitate the raising of the core temperature. As a result, person 90 can more comfortably wake up.

The first period, the second period, and the third period are, for example, set in advance and setting values are stored in storage 32. Furthermore, the first period, the second period, and the third period may be determined based on changes in the core temperature of person 90 who is sleeping. For example, controller 31 determines that the first period is a period of time starting from when person 90 goes to bed until when a rate of decrease of the core temperature becomes greater than or equal to a predetermined rate of decrease of the core temperature, determines that the second period is a period of time after the first period that lasts until when a rate of increase of the core temperature becomes greater than or equal to a predetermined rate of increase of the core temperature, and determines that the third period is a period of time after the second period that lasts until when person 90 wakes up. It should be noted that the periods of time with which the air temperatures are changed are not limited to three periods of time, namely, the first period, the second period, and the third period, and there may be two periods of time, and there may be four or more periods of time.

Furthermore, when control is performed based on both the season information and the temperature of the sleeping environment, controller 31 may adjust, based on the temperature of the sleeping environment, the air temperature determined based on the season information. For example, the higher the temperature of the sleeping environment, the lower the air temperature to which controller 31 sets the air temperature determined based on the season information is.

Furthermore, when controlling the air temperature of air blown by air blower 21 based on the temperature of the sleeping environment, controller 31 determines the air temperature such that, the lower the temperature of the sleeping environment, the higher the air temperature is set to, for example. Furthermore, even when controlling the air temperature of air blown by air blower 21 based on the temperature of the sleeping environment, as described above, controller 31 may change the air temperature over a plurality of periods of time by which the period of time starting from when person 90 goes to bed until when person 90 wakes up is divided.

Furthermore, for example, controller 31 determines the air temperature such that, the lower the temperature of the season or the lower the temperature of the sleeping environment, the higher the air temperature of the first period is set to. Accordingly, since the air temperature becomes higher in environments in which person 90 is more likely to feel cold, person 90 can more comfortably fall asleep.

Furthermore, in step S42, controller 31 may obtain, via obtainer 33, the extremity temperature of person 90 measured by extremity temperature measurer 56, and may change, based on the extremity temperature of person 90 obtained, the air temperature determined based on at least one of the season information or the temperature of the sleeping environment. For example, the higher the extremity temperature of person 90, the lower the air temperature, which is determined based on at least one of the season information or the temperature of the sleeping environment, to which controller 31 sets the air temperature is.

It should be noted that the air temperature may be set in an arbitrary manner by the user. Furthermore, when controller 31 does not use one of the season information or the temperature of the sleeping environment for controlling the air temperature, controller 31 need not obtain the one of the season information or the temperature of the sleeping environment in step S41 or step S42.

Next, another example of operation of sleep assistance system 10b according to the present variation will be described. FIG. 12 is a flowchart illustrating another example of operation of sleep assistance system 10b according to the present variation. Even in the other example of operation illustrated in FIG. 12, in the same manner as the example illustrated in FIG. 10, controller 31 controls the air temperature of air blown by air blower 21 by using temperature adjuster 22. It should be noted that controller 31 may perform the operations illustrated in FIG. 12 in parallel with the operations described with reference to FIG. 5.

First, person 90 that is trying to sleep enters bedding 80, and by the actuation of air blower 21 being turned on or the like, air blower 21 starts blowing air at a predetermined air volume at foot 91 of person 90. Then, controller 31 obtains, via obtainer 33, an extremity temperature of person 90 who is sleeping in bedding 80 that is measured by extremity temperature measurer 56 (step S51). For example, extremity temperature measurer 56 measures the extremity temperature of person 90 at a predetermined interval, and controller 31 causes a result of the extremity temperature measured at the predetermined interval to be stored in storage 32 as an extremity temperature history together with time information. The obtaining of the extremity temperature of person 90 performed by controller 31 is continued at the predetermined interval until person 90 wakes up. The predetermined interval is, for example, no less than 10 seconds and no more than 10 minutes.

Next, after a given period of time has elapsed starting from when person 90 goes to bed, controller 31 determines whether the extremity temperature of person 90 is decreasing (step S52). In the process in step S52 performed by controller 31, the same process as described in the above-mentioned step S15 is performed, for example. Accordingly, controller 31 can determine whether person 90 is feeling cold. In the process in step S52, in the same manner as step S15, a process may be performed to determine whether person 90 is feeling cold by using a detection result of bodily movement instead of the extremity temperature of person 90. Furthermore, in step S52, controller 31 may determine whether the extremity temperature of person 90 is decreasing and whether the rate of decrease of the extremity temperature is greater than or equal to a predetermined threshold value.

When it is determined that the extremity temperature of person 90 is decreasing (“Yes” in step S52), controller 31 then performs control to cause the air temperature of air blown by air blower 21 to be higher than the extremity temperature of person 90 (step S53). For example, controller 31 determines the air temperature of the air blown by air blower 21 to be “the extremity temperature of person 90+α (° C.)”, and, by using temperature adjuster 22, adjusts the air temperature of the air blown by air blower 21 to match the air temperature determined.

On the other hand, when it is determined that the extremity temperature of person 90 is not decreasing (“No” in step S52), controller 31 then performs control to cause the air temperature of air blown by air blower 21 to be lower than the extremity temperature of person 90 (step S54). For example, controller 31 determines the air temperature of the air blown by air blower 21 to be “the extremity temperature of person 90−β (° C.)”, and, by using temperature adjuster 22, adjusts the air temperature of the air blown by air blower 21 to match the air temperature determined.

By performing step S52 to step S54 in this manner, since air with an air temperature that is higher than the extremity temperature of person 90 is blown at an extremity of person 90 when person 90 is feeling cold, person 90 can be prevented from feeling discomfort while sleeping. Consequently, not only is it possible to facilitate the lowering of the core temperature by blowing air to cause the air to contact the extremity of person 90, but person 90 can more comfortably fall asleep.

Furthermore, in step S52 to step S54, instead of determining whether the extremity temperature of person 90 is decreasing, controller 31 may cause the air temperature to be higher than the extremity temperature when the extremity temperature of person 90 is less than or equal to a first threshold value, and may cause the air temperature to be lower than the extremity temperature when the extremity temperature of person 90 is greater than a second threshold value that is a value greater than or equal to the first threshold value. This also allows person 90 to more comfortably fall asleep. Furthermore, since the extremity temperature of person 90 is correlated with an amount of blood flow in the extremity of person 90, the degradation of heat-dissipation performance from the extremity can be inhibited by preventing the amount of blood flow from becoming excessively small, and the lowering of the core temperature can further be facilitated.

After step S53 or step S54, controller 31 determines whether a predetermined period of time has elapsed starting from the point in time when person 90 goes to bed (step S55). The length of the predetermined period of time is, for example, no less than two hours and no more than seven hours. When it is determined that the predetermined period of time has not elapsed starting from the point in time when person 90 goes to bed (“No” in step S55), controller 31 once again performs step S52 after a given period of time has elapsed. The given period of time is, for example, no less than one minute and no more than 30 minutes. In this manner, step S52 and step S53 or step S54 are repeatedly performed until the predetermined period of time has elapsed. Furthermore, instead of the operation of step S55, controller 31 may determine whether the core temperature of person 90 who is sleeping is increasing after a predetermined period of time has elapsed starting from the point in time when person 90 goes to bed. In this case, step S52 and step S53 or step S54 are repeatedly performed until the core temperature of person 90 who is sleeping begins increasing.

When it is determined that the predetermined period of time has elapsed starting from when person 90 goes to bed (“Yes” in step S55), controller 31 then performs control to cause the air temperature of air blown by air blower 21 to be higher than the extremity temperature of person 90 (step S56). Subsequently, air with an air temperature that is controlled in step S56 continues to be blown until person 90 wakes up and the actuation of air blower 21 is turned off. For example, controller 31 determines the air temperature of the air blown by air blower 21 to be “the extremity temperature of person 90+γ (° C.)”, and, by using temperature adjuster 22, adjusts the air temperature of the air blown by air blower 21 to match the air temperature determined. Accordingly, the air temperature of air blown by air blower 21 increases, over a period of time at a stage in which the core temperature increases as person 90 prepares to wake up, thereby making it possible to facilitate the raising of the core temperature. As a result, person 90 can more comfortably wake up.

α, β, and γ, as described above, are set by receiving input from the user, for example. Furthermore, α, β, and γ may be adjusted or determined based on at least one of the temperature of the sleeping environment of person 90 or the extremity temperature of person 90. For example, the lower the temperature of the season, the lower the temperature of the sleeping environment, or the lower the extremity temperature, the higher the values to which α and γ are set, and the lower the value to which β is set by controller 31.

As described above, in sleep assistance system 10b, controller 31 obtains at least one of the season information, the temperature of the sleeping environment of person 90, or the extremity temperature of person 90, and controls the air temperature of air blown by air blower 21 based on the at least one. Accordingly, the lowering of the core temperature is facilitated by blowing air at the extremity of person 90, while the likelihood of causing discomfort for person 90 during sleeping is reduced by controlling the air temperature, thereby enhancing the quality of sleep of person 90.

### Other Embodiments

While the sleep assistance systems according to the present disclosure have been described based on the embodiments (includes variations), the present disclosure is not limited to these embodiments. Forms obtained by various modifications to the embodiments that may be conceived by a person of ordinary skill in the art or forms obtained by combining elements in different embodiments, for as long as they do not depart from the essence of the present disclosure, may also be included within the scope of the present disclosure.

For example, in the above-mentioned embodiment, although controller 31 controls the air volume of air blower 21 based on the core temperature of person 90 who is sleeping, this example is not limiting. For example, an air volume that is appropriate for facilitating the lowering of the core temperature of person 90 may be verified in advance, and the air volume may be stored in storage 32. In this case, controller 31 controls air blower 21 at the air volume that is stored in storage 32. Furthermore, in this case, a portion of the operations of the above-mentioned step S12 and onward may be performed.

Furthermore, for example, in the above-mentioned embodiment, air blower 21 blows air at a foot of person 90 who is sleeping in bedding 80, but this example is not limiting. Air blower 21 may blow air at person 90 who is sleeping in something other than bedding 80, such as a seat in a vehicle, a chair for taking a nap, or the like.

Furthermore, for example, in the above-mentioned embodiment, air blower 21 blows air at foot 91 of person 90, but this example is not limiting. As an extremity of person 90, air blower 21 may blow air at a hand of person 90.

Furthermore, in the above-mentioned embodiment, the sleep assistance system is implemented as a plurality of devices, but may be implemented as a single device. For example, the sleep assistance system may be implemented as a single device in which the air blowing device and the control device are integrated with each other. Furthermore, when implementing the sleep assistance system as a plurality of devices, each of the elements included in the sleep assistance system may be assigned to the plurality of devices in any manner desired. Furthermore, the plurality of devices may communicate via a network, such as the Internet or the like.

Furthermore, in the above-mentioned embodiments, processes to be executed by a certain processor may be performed by another processor. Furthermore, the order of the plurality of processes may be changed, and the plurality of processes may be executed in parallel with each other.

For example, the processes described in the above-mentioned embodiment may be implemented by performing integrated processing using a single device, or may be implemented by performing decentralized processing using a plurality of devices. Furthermore, the processor that executes the above-mentioned program may be a single processor or a plurality of processors. In other words, integrated processing may be performed, and, alternatively, decentralized processing may be performed.

Furthermore, general and specific aspects of the present disclosure may be implemented as a system, a device, a method, an integrated circuit, a computer program, or a computer-readable recording medium such as a CD-ROM. Furthermore, general and specific aspects of the present disclosure may be implemented as any combination of a system, a device, a method, an integrated circuit, a computer program, or a recording medium. For example, the present disclosure may be implemented as a sleep assistance method executed by a computer, such as a control device. Furthermore, the present disclosure may be implemented as a program for causing a computer to execute the sleep assistance method, and may be implemented as a non-transitory computer-readable recording medium having recorded thereon such a program.

### INDUSTRIAL APPLICABILITY

The sleep assistance system according to the present disclosure is applicable to a system used while a person is sleeping.

1. A sleep assistance system comprising:
an air blower that blows air to cause the air to directly hit an extremity of a person; and
a controller that controls the air blown by the air blower to facilitate lowering of a core temperature of the person.

2. The sleep assistance system according to claim 1, wherein
the controller controls, based on the core temperature of the person, the air blown by the air blower.

3. The sleep assistance system according to claim 2, wherein
the controller controls the air blown by the air blower to cause a rate of decrease of the core temperature of the person to be greater than or equal to a predetermined threshold value.

4. The sleep assistance system according to claim 2, wherein
the controller obtains the core temperature of the person measured by a core temperature measurer, and controls the air blown by the air blower based on the core temperature obtained.

5. The sleep assistance system according to claim 2, wherein
the controller obtains a heart rate of the person, estimates the core temperature of the person based on the heart rate obtained, and controls the air blown by the air blower based on the core temperature estimated.

6. The sleep assistance system according to claim 3, wherein
the controller obtains an extremity temperature of the person, and controls an air volume of the air blower to maximize the air volume of the air blower to an extent that the extremity temperature of the person does not decrease.

7. The sleep assistance system according to claim 1, wherein
the controller controls an air volume of the air blower to cause the air volume during a predetermined period of time starting from when the person goes to bed to be larger than the air volume of the air blower when the person wakes up.

8. The sleep assistance system according to claim 1, wherein
the controller obtains bodily sensation information that indicates a bodily sensation of the person, and controls the air blown by the air blower based on the bodily sensation information.

9. The sleep assistance system according to claim 8, wherein
the bodily sensation information indicates whether the person is feeling cold.

10. The sleep assistance system according to claim 8, wherein
the bodily sensation information is an extremity temperature of the person.

11. The sleep assistance system according to claim 8, wherein
the bodily sensation information is a detection result of bodily movement of the person.

12. The sleep assistance system according to claim 1, further comprising:
a temperature adjuster that heats the air blown by the air blower.

13. The sleep assistance system according to claim 1, wherein
the controller obtains a temperature of a sleeping environment of the person, and controls the air blown by the air blower based on the temperature of the sleeping environment obtained.

14. The sleep assistance system according to claim 1, wherein
the controller controls the air blown by the air blower based on season information.

15. The sleep assistance system according to claim 1, wherein
the controller obtains an extremity temperature of the person, and controls the air blown by the air blower based on the extremity temperature of the person obtained.

16. The sleep assistance system according to claim 1, wherein
the extremity is a foot of the person.

17. The sleep assistance system according to claim 16, wherein
the person is sleeping in bedding, and
the air blower blows air at the foot that is surrounded by the bedding.

18. The sleep assistance system according to claim 17, wherein
the bedding includes:
under bedding that is disposed below the person; and
over bedding that is disposed above the person, and
a portion of the over bedding is fixed to the under bedding in a region in which the foot of the person is located.

19. The sleep assistance system according to claim 16, wherein
when the person who is sleeping is viewed from above, the air blower blows air at the foot from a direction intersecting a head-to-toe height direction of the person.

20. The sleep assistance system according to claim 1, wherein
the extremity is disposed in a space covered by bedding,
the air blower includes:
an air blowing unit that generates a flow of air; and
an air duct through which the flow of air generated by the air blowing unit passes, and
an air outlet of the air duct is disposed in a predetermined position in the space covered by the bedding to cause the flow of air that exits the air outlet to directly hit the extremity.
